(19) United States
(12) Patent Application Publication
(10) Pub. No.: US 2022/0088257 A1
(43) Pub. Date: Mar. 24, 2022

(54) ATOMIZATION DEVICE AND AROMA DIFFUSER

(71) Applicant: Puzhen Life Co., Inc., Shatin (HK)

(72) Inventor: Andy LEE, Shatin (HK)

(73) Assignee: Puzhen Life Co., Inc., Shatin (HK)

(21) Appl. No.: 17/087,679

(22) Filed: Nov. 3, 2020

(30) Foreign Application Priority Data
Sep. 21, 2020 (CN) 202022087146.8

(51) Int. Cl. A61L 9/14

(52) U.S. Cl. CPC A61L 9/14; A61L 2209/134

(57) ABSTRACT

The present application provides an atomization device and an aroma diffuser. The atomization device includes: an oil supply structure including an oil storage bottle containing essential oil and an oil delivery pipe connected to the oil storage bottle, the oil storage pipe having a liquid outlet for the essential oil to flow out; a gas delivery structure for generating an atomized airflow; and an atomization structure including a first pipe section, a second pipe section and a third pipe section that are sequentially connected, an end of the first pipe section away from the second pipe section is connected to the gas delivery structure to receive the atomized airflow, the second pipe section is provided with an atomization port and in communication with the liquid outlet, and the third pipe section having a pipe diameter gradually increased in a direction away from the second pipe section.

### CROSS-REFERENCE TO THE RELATED APPLICATION

Pursuant to 35 U.S.C. § 119 and the Paris Convention Treaty, this application claims the benefit of Chinese Patent Application No. 202022087146.8 filed Sep. 21, 2020, the contents of which are incorporated herein by reference.

### TECHNICAL FIELD

The present application relates to the technical field of aromatherapy atomization, and more particularly to an atomization device and an aroma diffuser.

### BACKGROUND

The atomization device of the existing diffuser (e.g., the patent application No. 201880000924.8, entitled “essential oil reflux atomizer”) includes an air pump, an air nozzle, an oil storage bottle and an oil nozzle. The air nozzle abuts against the oil nozzle, the air pump drives air out through the air nozzle, and a negative pressure is formed at the air nozzle. The essential oil in the oil storage bottle is sprayed out from an oil nozzle under the action of the negative pressure and then being mixed and atomized with the air. There is a problem of large atomized particles being existed in this solution, which thereby affects the atomization effect.

### SUMMARY

An object of the present application is to overcome the above-mentioned shortcomings existed in the prior arts, an atomization device and an aroma diffuser are provided, which aims to improve the atomization effect of the atomization device.

In order to achieve the foregoing objectives, embodiments of the present application provide the following t

### Technical Solutions

An atomization device, including:

an oil supply structure which includes an oil storage bottle containing essential oil and an oil delivery pipe connected to the oil storage bottle, where the oil storage pipe has a liquid outlet for the essential oil to flow out;

a gas delivery structure which is configured for generating atomized airflow;

an atomization structure which includes a first pipe section, a second pipe section and a third pipe section that are sequentially connected, where an end of the first pipe section away from the second pipe section is connected with the gas delivery structure to receive the atomized airflow, the second pipe section is provided with an atomization port and in communication with the liquid outlet, and the third pipe section having a pipe diameter gradually increased in a direction away from the second pipe section;

where the atomized airflow generated by the gas delivery structure enters the atomization structure and forms a negative pressure at the atomization port that forces the essential oil to flow into the second pipe section, so that the essential oil is atomized at the second pipe section and sprayed out through the third pipe section.

Optionally, the first pipe section includes a connection portion and a constriction portion, the connection portion is connected to the gas delivery structure, and the constriction portion has a pipe diameter gradually decreased from the connection portion to the second pipe section.

Optionally, the first pipe section further includes an extension portion disposed between the constriction portion and the second pipe section, and the extension portion is provided with a same pipe diameter.

Optionally, the pipe diameter of the extension portion is smaller than the pipe diameter of the second pipe section.

Optionally, the atomization structure further includes a cavity expansion section connected to the third pipe section, and the cavity expansion section is provided with a counterbore on a surface away from the third pipe section, and a through hole in communication with the third pipe section and an annular groove arranged around the through hole are provided at a bottom of the counterbore.

An aroma diffuser which includes the atomization device as described above.

Optionally, the aroma diffuser has a diffusion cavity and a mist outlet that is in communication with the diffusion cavity and outside, the diffusion cavity is in communication with the third pipe section; the oil storage bottle is located below the diffusion cavity and in communication with the diffusion cavity, the oil delivery pipe inserted into a mouth of the oil storage bottle has a gap with the mouth of the oil storage bottle.

Optionally, the oil storage bottle has an oil storage chamber containing the essential oil, and the aroma diffuser further includes a leak-proof tube that closes the mouth of the oil storage bottle and extends into a bottom of the oil storage chamber. The anti-leakage pipe is in communication with the diffusion cavity and the oil storage chamber, and the anti-leakage pipe inserted into the oil delivery pipe has a gap with the oil delivery pipe.

Optionally, the leak-proof tube includes an upper section, a middle section and a lower section that are sequentially connected from top to bottom, and the upper section has a pipe diameter larger than that of the lower section.

Optionally, the aroma diffuser has a diffusion cavity and a mist outlet that is in communication with the diffusion cavity and the outside; the oil storage bottle has an oil storage chamber containing the essential oil, the oil storage chamber is located below the diffusion cavity and in communication with the diffusion cavity, a nozzle of the third pipe section is downward so that the atomized essential oil enters the oil storage chamber downward.

The atomization device and diffuser provided in the present application can reduce the particle size of essential oil after atomization and improve the atomization effect.

### BRIEF DESCRIPTION OF DRAWINGS

In order to illustrate the technical solutions of embodiments of the present application more clearly, the drawings used in the description of the embodiments will be briefly introduced hereinbelow. Obviously, the drawings in the following description are some embodiments of the present application, and for persons skill in the art, other drawings may also be obtained on the basis of these drawings without any creative work.

FIG. 1 is a first schematic diagram of a connection between an oil supply structure and an atomization structure in accordance with an embodiment of the present application;

FIG. 2 is a second schematic diagram, a cross-sectional view, of the connection between the oil supply structure and the atomization structure in accordance with an embodiment of the present application;

FIG. 3 is a cross-sectional view of the atomization structure in accordance with an embodiment of the present application;

FIG. 4 is a first schematic diagram, a cross-sectional view, of an aroma diffuser in accordance with an embodiment of the present application;

FIG. 5 is a schematic diagram of essential oil and gas flows in the structure of FIG. 4;

FIG. 6 is a second schematic diagram, a cross-sectional view, of an aroma diffuser in accordance with an embodiment of the present application;

FIG. 7 is the third schematic diagram, a cross-sectional view, of an aroma diffuser in accordance with an embodiment of the present application; and

FIG. 8 is a fourth schematic diagram, a cross-sectional view, of an aroma diffuser in accordance with an embodiment of the present application.

Among them, reference signs in the figures are listed below:

10 oil supply structure; 11 oil storage bottle; 101 oil storage chamber; 12 oil delivery pipe; 121 oil delivery section; 122 connection section; 20 gas delivery structure; 21 air pump; 22 gas delivery pipe; 30 atomization structure; 31 first pipe section; 311 connection portion; 312 constriction portion; 313 extension portion; 32 second pipe section; 33 third pipe section; 34 cavity expansion section; 301 counterbore; 302 through hole; 303 groove; 401 diffusion cavity; 402 mist outlet; 50 leak-proof tube.

### DETAILED DESCRIPTION OF THE EMBODIMENTS

In order to make technical problems to be solved by the present application, technical solutions and beneficial effects more comprehensible, the following further describes the present application in detail with reference to the drawings and embodiments. It should be understood that the specific embodiments described herein are merely used to explain the present application, and do not intend to limit the present application.

It should be noted that when an element is referred to as being “fixed to” or “disposed on” another element, it can be directly or indirectly on the other element. When an element is referred to as being “connected to” another element, it can be directly or indirectly connected to the other element.

It should be understood that the terms “length”, “width”, “upper”, “lower”, “front”, “rear”, “left”, “right”, “vertical”, “horizontal”, “top”, “bottom”, “inner”, “outer”, etc. indicate the orientation or positional relationship based on the orientation or positional relationship shown in the drawings, and are merely for the convenience of describing this application and simplifying the description, rather than indicating or implicitly referring to a device or element must have a specific orientation, be constructed and operated in a specific orientation, and therefore cannot be understood as a limitation of the present application.

In addition, the terms “first” and “second” are merely used for illustrative purposes, and cannot be understood as indicating or implying relative importance or implicitly indicating the number of indicated technical features. Thus, the features defined with “first” and “second” may explicitly or implicitly include one or more of these features. In the description of the present application, “a plurality of” means two or more than two, unless otherwise specifically defined.

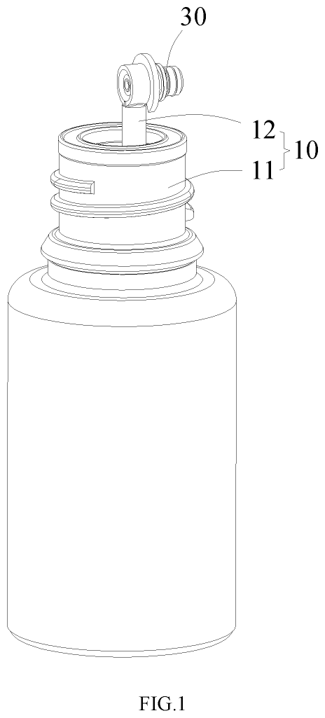

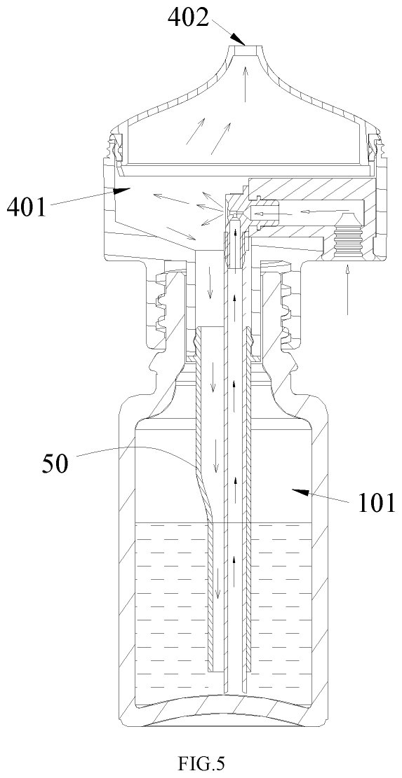

Please refer to FIGS. 1 to 9, an atomization device and an aroma diffuser having the atomization device provided by the present application will now be exemplified. FIGS. 5 and 7-9 show four schematic diagrams of the aroma diffuser having the atomization device.

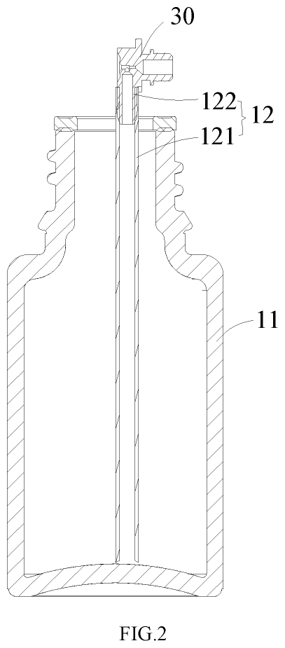

Please refer to FIGS. 2, 4 and 5, the atomization device includes an oil supply structure 10, a gas delivery structure 20 and an atomization structure 30.

The oil supply structure 10 includes an oil storage bottle 11 containing essential oil and an oil delivery pipe 12 connected to the oil storage bottle 11, and the oil delivery pipe 12 has a liquid outlet for the essential oil to flow out.

The gas delivery structure 20 is configured to generate atomized airflow. In the aroma diffuser structure shown in any one of FIGS. 5 and 7 to 9, the gas delivery structure 20 includes an air pump 21 and a gas delivery pipe 22. The air pump 21 drives air into the gas delivery pipe 22 to form a high-speed moving atomized airflow. It can be understood that the gas delivery pipe 22 may be a single structure, or may be formed by connecting multiple parts; and it may be a flexible pipe or a structure with an airflow channel, which is not limited here.

The atomization structure 30 includes a first pipe section 31, a second pipe section 32 and a third pipe section 33 that are sequentially connected. An end of the first pipe section 31 away from the second pipe section 32 is connected to the gas delivery structure 20 to receive the atomized airflow. The second pipe section 32 is provided with an atomization port communicating with the liquid outlet, and the pipe diameter of the third pipe section 33 is arranged to be gradually increased in a direction away from the second pipe section 32.

Among them, the atomized airflow generated by the gas delivery structure 20 enters the atomization structure 30 and forms a negative pressure at the atomization port that forces the essential oil into the second pipe section 32. The essential oil in the oil storage bottle 11 enter the second pipe section 32 through the liquid outlet under the negative pressure and being mixed with the atomized airflow, where the atomized airflow shears the essential oil to make it in an atomized state, and is driven by the air pump 21 to spray out through the third pipe section 33.

The essential oil is mixed and atomized in the second pipe section 32 with the atomized airflow for the first time, and then further being mixed and atomized in the third pipe section 33 as the pipe diameter increases, and finally, when it leaves the third pipe section 33 and enters the outside air or cavity with the further expansion of the space, it can be atomized further again. In view of the above, the essential oil can be atomized three times to form smaller particles, thereby improving the atomization effect of the essential oil.

It should be noted that an arrangement of the second pipe section 32 can provide a mixing space boundary for the essential oil and the atomized airflow. The internal space of the second pipe section 32 provides a gas flow channel for the mixed atomized airflow and the essential oil; with such a structure, the outside air is prevented from entering the internal space of the second pipe section 32, thereby preventing the outside air from entering the gas flow channel to generate local turbulence and block the velocity of the gas flowing forward. Under the same air pump 21, the arrangement of the second pipe section 32 compared with the design having no the second pipe section 32, it can increase the overall flow rate of the gas in the second pipe section 32, so that the essential oil is more easily broken up into smaller particles and the atomization effect is improved.

In summary, the atomization device provided in this embodiment can reduce the particle size of the essential oil after atomization and improve the atomization effect.

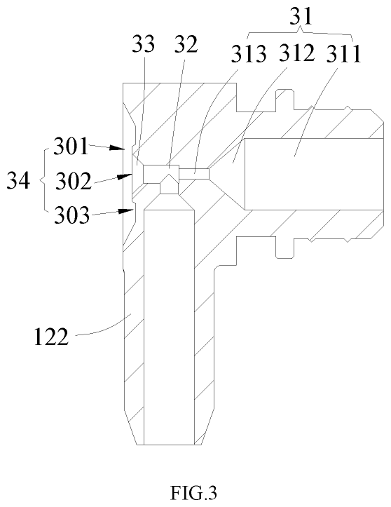

In another embodiment of the present application, referring to FIG. 3, the first pipe section 31 includes a connection portion 311 and a constriction portion 312. The connection portion 311 is connected to the gas delivery structure 20. Specifically, the connection portion 311 is connected to the gas delivery pipe 22, the pipe diameter of the constriction portion 312 is arranged to be gradually decreased from the connection portion 311 to the second pipe section 32. Two ends of the constriction portion 312 have a first diameter and a second diameter, respectively, and the end located at the junction of the constriction portion 312 and the connection portion 311 has the first diameter. The first diameter is greater than the second diameter. The atomized airflow enters the connection portion 311 from the gas delivery pipe 22 and then enters the second pipe section 32 through the constriction portion 312. Because the pipe diameter of the constriction portion 312 is gradually decreased, the atomized airflow converges at the constriction portion 312 and accelerates to flow to the second pipe section 32, in other words, the arrangement of the constriction portion 312 can increase the velocity of the atomized airflow entering the second pipe section 32 or the liquid outlet, so as to increase the negative pressure at the liquid outlet and further improve the atomization effect of the essential oil.

Preferably, the ratio of the first diameter to the second diameter is no less than 5. Those skilled in the art can further take the ratio as: 5.0, 5.2, 5.4, 5.5, 5.8, 6.0, 6.3, 6.5, 7.0, 8.0, 8.5, 8.8, 9.0, etc., which is not particularly limited here.

In another embodiment of the present application, please refer to FIG. 3, the first pipe section 31 further includes an extension portion 313 provided between the constriction portion 312 and the second pipe section 32, and the extension portion 313 is provided with equal pipe diameters. The pipe diameter of the extension portion 313 is equal to the second diameter. The arrangement of the extension portion 313 facilitates the formation of a vortex of the atomized airflow at the constriction portion 312 and moves to the second pipe section 32 via the extension portion 313, thereby facilitating the mixing of the atomized airflow and the essential oil and improving the atomization effect.

In another embodiment of the present application, please refer to FIG. 3, the pipe diameter of the extension portion 313 is smaller than the pipe diameter of the second pipe section 32. In combination with the foregoing, the extension portion 313 is provided with a smaller pipe diameter to facilitate increasing the velocity of the atomized airflow entering the second pipe section 32. The diameter of the second pipe section 32 is larger than that of the extension portion 313, so as to facilitate the essential oil entering the second pipe section 32 and being mixed with the atomized airflow.

Preferably, the ratio of the pipe diameter of the second pipe diameter 32 to the pipe diameter of the extension portion 313 is no less than 1.2. Those skilled in the art can further take the ratio as: 1.2, 1.3, 1.5, 1.6, 1.8, 2.0, etc., which is not particularly limited here.

The diameter of the second pipe section 32 is larger than the diameter of the liquid outlet. On the one hand, the internal space of the second pipe section 32 is a space for mixing essential oil and atomized airflow, the essential oil enters the second pipe section 32 through the liquid outlet, and the diameter of the second pipe section 32 is larger than that of the liquid outlet to provide sufficient mixing space; On the other hand, the larger the ratio of the diameter of the second pipe section 32 to the diameter of the liquid outlet, the smaller the proportion of essential oil occupying the mixing space, and the influence of essential oil on the flow of atomized airflow is reduced, which ensures that the atomized airflow and the essential oil can be mixed and continues to move forward.

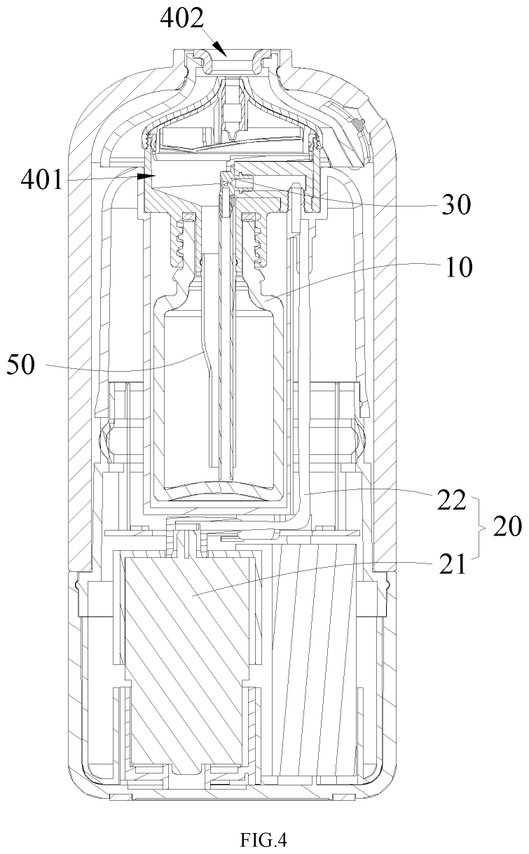

In this embodiment, referring to FIGS. 2 and 4, the oil delivery pipe 12 includes an oil delivery section 121 and a connection section 122 detachably connected. The connection section 122 is connected to the second pipe section 32. Preferably, the connection section 122 is disposed integrally with the atomization structure 30. The oil delivery section 121 is plugged into the connection section 122 and used to guide essential oil into the connection section 122. This arrangement ensures the tightness of the connection between the oil delivery pipe 12 and the second pipe section 32. The oil delivery section 121 and the connection section 122 are plug-connected to improve the convenience of production and assembly.

In another embodiment of the present application, referring to FIG. 4, the atomization structure 30 further includes a cavity expansion section 34 connected to the third pipe section 33. The cavity expansion section 34 is provided with a counterbore 301 on a surface away from the third pipe section 33, and a through hole 302 communicating with the third pipe section 33 and an annular groove 303 arranged around the through hole 302 are provided at the bottom of the counterbore 301. the atomized gas is further diffused when reaches the cavity expansion section 34 due to the increase in the space of the cavity expansion section 34. In addition, the arrangement of the groove 303 facilitates the diffusion of gas from the through hole 302 to the groove 303, which breaks a laminar flow state of the gas and causes small vortices in the flow field to further improve the diffusion effect of the essential oil.

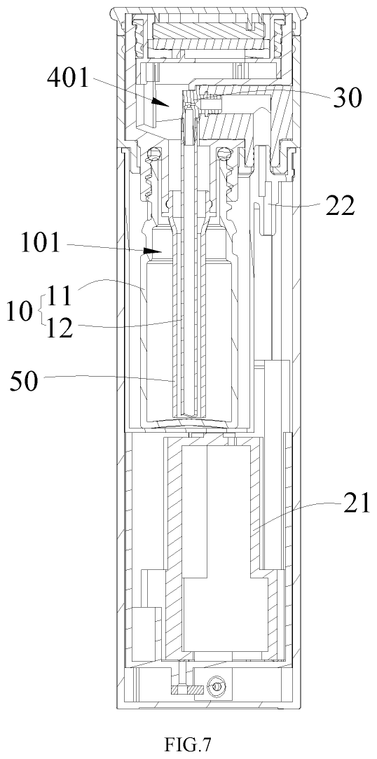

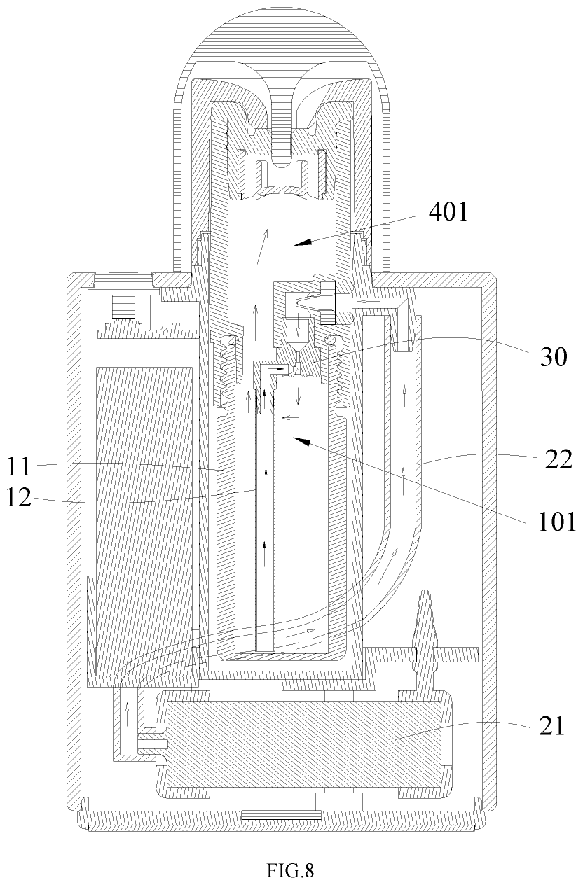

In another embodiment of the present application, please refer to FIG. 5, FIG. 7 or FIG. 8. the aroma diffuser has a diffusion cavity 401 and a mist outlet 402 that communicates with the diffusion cavity 401 and the outside. the diffusion cavity 401 is in communication with the third pipe section 33; The oil storage bottle 11 is located below the diffusion 401 and communicates with the diffusion cavity 401, and the oil delivery pipe 12 is inserted into a mouth of the oil storage bottle 11 leaving a gap with the mouth of the oil storage bottle 11.

FIG. 5 shows the flow paths of the essential oil and the atomized airflow. In the figures, the outlined arrows indicating the flow direction of the atomized airflow, the solid arrows indicating the movement direction of the essential oil, and the 30-degree angle arrows indicating the flow direction of the essential oil and the atomized airflow after being mixed.

The atomized airflow enters the atomization structure 30 through the gas delivery pipe 22, the essential oil is mixed with the atomized airflow in the second pipe section 32 and sprayed toward the diffusion cavity 401 in the third pipe section 33. There are large particles and small particles of essential oil in the atomized gas, as for the small particles of lighter mass, they may diffuse from the mist outlet 402 to the outside as the atomized airflow upwards. For large particles of heavier mass, they fall into the diffusion cavity 401 and reach the mouth of the oil storage bottle 11 along the wall of the diffusion cavity 401 and fall back into the oil storage bottle 11 to realize a recovery of essential oil and reduce a waste of essential oil.

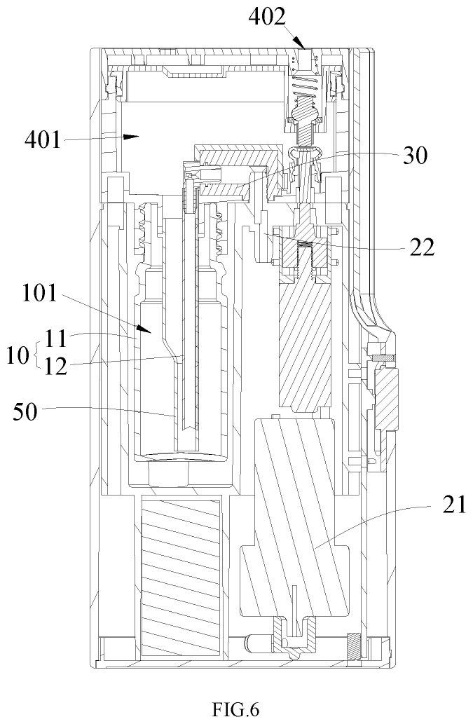

In another embodiment of the present application, please refer to FIG. 6, the oil storage bottle 11 has an oil storage chamber 101 containing essential oil, and the aroma diffuser further includes a mouth that closes the oil storage bottle 11 and an anti-leakage pipe 50 that extends into the bottom of the oil storage chamber 101. The anti-leakage pipe 50 is in communication with the diffusion cavity 401 and the oil storage chamber 101, the oil delivery pipe 12 may be inserted into the anti-leakage pipe 50 with a gap between the oil delivery pipe 12 and the anti-leakage pipe 50. The essential oil of large particles are returned to the oil storage chamber 101 through the leak-proof tube 50. The aroma diffuser provided in this embodiment may be a handheld type or a vertical type. In a normal state of use, the oil storage bottle 11 is placed in a forward direction, with its mouth facing upwards. When the aroma diffuser is inverted or tilted, only the liquid level of the essential oil in the oil storage bottle 11 is higher than the port of the leak-proof tube 50, the essential oil can enter the leak-proof tube 50 and may flow out from the mist outlet 402. Therefore, the arrangement of the anti-leakage tube 50 increases the difficulty of essential oil leakage.

It can be understood that appropriately increasing the length of the leak-proof tube 50, or in other words, shortening the distance between the leak-proof tube 50 and the lower wall of the oil storage chamber 101, can further increase the difficulty of essential oil leakage.

In the structure shown in FIG. 6, the leak-proof tube 50 includes an upper section, a middle section and a lower section that are sequentially connected. The upper section has a larger pipe diameter than the lower section. The leak-proof tube 50 has a larger pipe diameter at a nozzle (upper section) of the oil storage bottle 11 so as to facilitate the fall of the essential oil, the lower section has a smaller pipe diameter to increase the difficulty of essential oil leakage.

FIG. 9 shows another diffuser, and the moving path of the essential oil after atomization is different from the schemes of FIGS. 5, 7 and 8. Please refer to FIG. 9, the aroma diffuser has a diffusion cavity 401 and a mist outlet 402 that communicates with the diffusion cavity 401 and outside; the oil storage bottle 11 has an oil storage chamber 101 containing essential oil, and the oil storage chamber 101 is located below the diffusion cavity 401 and in communication with the diffusion cavity 401, and a nozzle of the third pipe section 33 is downward so that the atomized essential oil is sprayed downward into the oil storage chamber 101.

In the structure illustrated in the figures, the second pipe section 32 extends up and down, and the oil delivery pipe 12 is a bent pipe so that its outlet is perpendicular to the extension direction of the second pipe section 32. The essential oil enters the second pipe section 32 from the liquid outlet and mixes with the atomized airflow, then enters the oil storage chamber 101 downward from the third pipe section 33, and then enters the diffusion cavity 401 from the mouth of the oil storage bottle 11, and then diffuses from the mist outlet 402 to the outside. Since the atomized gas of the essential oil first enters the oil storage chamber 101 and then enters the diffusion cavity 401, the path of the gas movement is increased, and the difficulty of moving up to the mist outlet 402 is also increased, such that the larger liquid particles in the atomized gas fall into the oil storage chamber 101, thereby achieving the recovery of essential oil, and reducing the waste of essential oil.

The foregoing description are merely preferred embodiments of the present application, and are not intended to limit the present application. Any modifications, equivalent substitutions and improvements made within the spirit and principles of this disclosure should be understood as being included within the scope of the present application.

1. An atomization device, comprising:
an oil supply structure comprising an oil storage bottle containing essential oil and an oil delivery pipe connected to the oil storage bottle, the oil storage pipe having a liquid outlet for the essential oil to flow out;
a gas delivery structure configured for generating an atomized airflow;
an atomization structure comprising a first pipe section, a second pipe section and a third pipe section being sequentially connected, an end of the first pipe section away from the second pipe section being connected to the gas delivery structure to receive the atomized airflow, the second pipe section provided with an atomization port and in communication with the liquid outlet, and the third pipe section having a pipe diameter gradually increased in a direction away from the second pipe section;
wherein the atomized airflow generated by the gas delivery structure enters into the atomization structure and forms a negative pressure at the atomization port forcing the essential oil to flow into the second pipe section, so that the essential oil is atomized at the second pipe section and sprayed out through the third pipe section.

2. The atomization device according to claim 1, wherein the first pipe section comprises a connection portion and a constriction portion, the connection portion is connected to the gas delivery structure, and the constriction portion has a pipe diameter gradually decreased from the connection portion to the second pipe section.

3. The atomization device according to claim 2, wherein the first pipe section further comprises an extension portion disposed between the constriction portion and the second pipe section, and the extension portion has a same pipe diameter.

4. The atomization device according to claim 3, wherein the pipe diameter of the extension portion is smaller than the pipe diameter of the second pipe section.

5. The atomization device according to claim 1, wherein the atomization structure further comprises a cavity expansion section connected to the third pipe section, and the cavity expansion section is provided with a counterbore on a surface away from the third pipe section, and a through hole in communication with the third pipe section and an annular groove arranged around the through hole are provided at the bottom of the counterbore.

6. An aroma diffuser having an atomization device which comprising:
an oil supply structure comprising an oil storage bottle containing essential oil and an oil delivery pipe connected to the oil storage bottle, the oil storage pipe having a liquid outlet for the essential oil to flow out;
a gas delivery structure configured for generating an atomized airflow;
an atomization structure comprising a first pipe section, a second pipe section and a third pipe section being sequentially connected, an end of the first pipe section away from the second pipe section being connected to the gas delivery structure to receive the atomized airflow, the second pipe section provided with an atomization port and in communication with the liquid outlet, and the third pipe section having a pipe diameter gradually increased in a direction away from the second pipe section;
wherein the atomized airflow generated by the gas delivery structure enters into the atomization structure and forms a negative pressure at the atomization port forcing the essential oil to flow into the second pipe section, so that the essential oil is atomized at the second pipe section and sprayed out through the third pipe section.

7. The aroma diffuser according to claim 6, wherein the aroma diffuser has a diffusion cavity and a mist outlet that is in communication with the diffusion cavity and outside, and the diffusion cavity is in communication with the third pipe section; the oil storage bottle is located below the diffusion cavity and in communication with the diffusion cavity, and the oil delivery pipe is inserted into a mouth of the oil storage bottle with a gap between the mouth of the oil storage bottle and the oil delivery pipe.

8. The aroma diffuser according to claim 7, wherein the oil storage bottle has an oil storage chamber containing the essential oil, and the aroma diffuser further comprises a leak-proof tube that closes the mouth of the oil storage bottle and extends into a bottom of the oil storage chamber, wherein the leak-proof tube is in communication with the diffusion cavity and the oil storage chamber, and the leak-proof tube is inserted into the oil delivery pipe with a gap between oil delivery pipe and the leak-proof tube.

9. The aroma diffuser according to claim 8, wherein the leak-proof tube comprises an upper section, a middle section and a lower section sequentially connected from top to bottom, and the upper section has a pipe diameter larger than that of the lower section.

10. The aroma diffuser according to claim 6, wherein the aroma diffuser has a diffusion cavity and a mist outlet that is in communication with the diffusion cavity and outside; the oil storage bottle has an oil storage chamber containing the essential oil, wherein the oil storage chamber is located below the diffusion cavity and in communication with the diffusion cavity, and a nozzle of the third pipe section is downward so that the atomized essential oil enters the oil storage chamber downward.

11. The aroma diffuser according to claim 6, wherein the first pipe section comprises a connection portion and a constriction portion, the connection portion is connected to the gas delivery structure, and the constriction portion has a pipe diameter gradually decreased from the connection portion to the second pipe section.

12. The aroma diffuser according to claim 11, wherein the first pipe section further comprises an extension portion disposed between the constriction portion and the second pipe section, and the extension portion has a same pipe diameter.

13. The aroma diffuser according to claim 12, wherein the pipe diameter of the extension portion is smaller than the pipe diameter of the second pipe section.

14. The aroma diffuser according to claim 6, wherein the atomization structure further comprises a cavity expansion section connected to the third pipe section, and the cavity expansion section is provided with a counterbore on a surface away from the third pipe section, and a through hole in communication with the third pipe section and an annular groove arranged around the through hole are provided at the bottom of the counterbore.
